(19) United States
(12) Patent Application Publication
(10) Pub. No.: US 2004/0049216 A1
(43) Pub. Date: Mar. 11, 2004

(54) Device for perforating system tissue and its use

(76) Inventor: Rudolf M. Verdaasdonk, Houten (NL)

Correspondence Address: BROWN, MARTIN, HALLER & MCCLAIN LLP, 1660 UNION STREET, SAN DIEGO, CA, 92101-2926, US

(21) Appl. No.: 10/381,096

(22) PCT Filed: Sep. 24, 2001
(86) PCT No.: PCT/EP01/11128

(30) Foreign Application Priority Data
Sep. 22, 2000 (EP) 002032894
Sep. 22, 2000 (US) 60234737

(51) Int. Cl. A61B 17/32

(52) U.S. Cl. 606/169

(57) ABSTRACT

Device for perforating tissue, especially for transmyocardial revascularisation, comprising an ultrasonic generator coupled to an attachable solid needle.

The present invention relates to a device for perforating tissue and to the use of said device.

Transmyocardial revascularisation (TMR) has been shown effective in reducing angina and increasing exercise tolerance in patients suffering from an end-stage coronary heart disease who do not respond to medication and are unsuitable for standard revascularisation techniques. TMR is a method for revascularising the myocardial tissue through stimulation of angiogenesis and/or arteriogenesis by perforating the myocardial tissue such that small channels are created in the myocardium.

It is known to create such channels in the myocardium using high power pulsed lasers, resulting in an effective relief in angina. The laser energy induces an explosive vaporisation of the tissue associated with mechanical rupture and thermal injury up to several hundreds of micrometers into the tissue. The channels created typically have diameters between 1 and 2 mm, and a length of 5 to 30 mm. Relief of angina of 2 classes with an acceptable mortality (5-10%) and morbidity (20-30%) is achieved in the majority of patients treated by such transmyocardial laser revascularisation (TMLR). Based on experience in over 10,000 patients, TMLR has been shown to be an effective and safe procedure resulting in a significant improvement in the quality of life for a carefully selected patient group suffering from end-stage coronary heart disease.

Although TMLR is approved by the FDA and other regulatory agencies, and surgical TMLR can be a valuable adjunct to coronary artery bypass grafting (CABG) procedures to induce revascularisation of the myocardium which can not be sufficiently supplied with blood with grafts, the adoption rate by surgeons has been slow. The costs of the equipment, the safety requirements for using lasers, as well as the poorly understood mechanism of TMLR, have been a barrier to the general use of the TMLR procedure.

The present invention provides a device that induces similar tissue effects as lasers, but which is more pratical to handle in surgery, and which is much more cost effective than the existing surgical laser TMR systems.

The present invention thus provides a device for perforating tissue, comprising an ultrasonic vibration generator coupled to an attachable solid needle. The device according to the invention creates channels in the myocardium comparable to lasers through a modification of existing ultrasonically induced cavitation technology.

Surgical ultrasonic devices are known, particularly in neurosurgery and liver surgery, for resecting soft and hard tissues selectively in favor of elastic tissues like blood vessels, thus enabling the removal of tumours with a minimal loss of blood. These devices comprise an ultrasonic vibration generator in a surgical handpiece which has an attachable hollow titanium needle, enveloped by a plastic sheath through which saline is flushed which acts a lubricant and cools the needle. Through the hollow needle shaft the irrigation fluid is sucked away together with dissected tissue particles. The aspirated tissue thus may be examined by histopathology.

The conventional hollow aspirating needles as described above can, however, not be used for TMR since their diameter can not be reduced sufficiently such that small channels can be created in tissue, i.e with diameters between approximately 0.5 and 1 mm or less. The transmural channels should occlude at the surface of the epicardium to prevent excessive blood loss. If the diameter is larger than 1 mm this will not occur. If the channels are smaller than 0.5 mm the response to the TMR might be less effective.

Although surgical ultrasound needles typically are made of strong materials such as titanium, the reduction in diameter to less than about 1.7 mm results in a needle which can not withstand the large-amplitude vibrations necessary to create channels in tissue.

According to the present invention the device preferably comprises a tapered solid needle. The shape of the taper is preferably designed to match the ideal curvature to transfer ultrasonic waves through the needle and to obtain a standing wave in the needle. Such designs are well-known in the surgical ultrasound field. The special tapered shape of the needle enables longitudinal oscillation of the needle which will result in an amplification of the wave amplitude at the tip of the needle.

The device according to the present invention is for example activated by electrical enery, which induces the ultrasonic vibration generator, such as piezo-electric or ferromagnetic transducers, to expand and contract. The ultrasonic vibration generator generates vibration waves with typical frequencies between 20 and 60 kHz. The ultrasonic vibration frequency preferably is 23 or 35 kHz. Most of ultrasound generators available on the market have frequencies near 23 and 35 kHz. Thus, there are several generators available to drive the needle. These vibration waves are coupled to and passed through the needle. Due to its tapered shape, the initial longitudinal vibration amplitude of the vibration wave is amplified in the needle. Thus an amplitude of around 10 μm at the proximal end of the needle may be amplified to 350 μm expansion and contraction at the distal tip of the needle, as a result of which cavitation effects are induced at the tip of the device.

Cavitation, as used in this application, refers to the formation of gas or vapor-filled bubbles caused by sudden reductions in pressure in a fluid-like environment (water, blood, organic tissue), induced by a fast-moving object. In front of this object liquid is compressed, while at the back of the object a gap is created. The liquid is too slow to fill the gap, creating a near-vacuum. Cavitation typically occurs at the trailing edge of ship propellers and in liquids subject to ultrasonic waves of high power densities. By inducing cavitation effects the myocardial tissue can be ruptured, fragmented and vaporised. Thermal effects and shock waves may furthermore induce the release of particular cellular factors, and, additionally, the biochemical effects associated with the formation of free radicals may have potential beneficial effects in transmyocardial revascularisation.

The device according to the invention is very user-friendly, and easily implementable. During cardiac surgery, the needle tip is placed on the myocardial surface and, while activated, can be pushed gently into the tissue, thus perforating the tissue. There are no requirements for safety in the operating rooms. The device furthermore is less expensive and requires lower maintenance and service costs compared to the laser systems currently used for TMR.

In a preferred embodiment, the needle is machined from titanium, which is a very strong and reliable material.

In order to prevent large amounts of blood loss the tip diameter of the needle is approximately 1 mm.

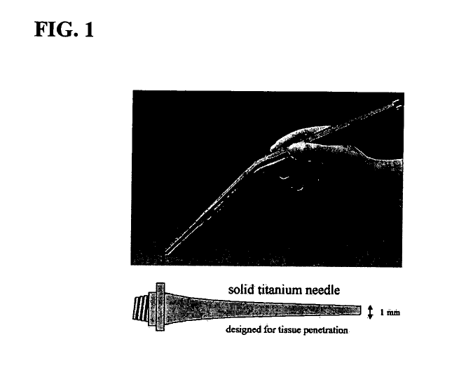

In a preferred embodiment of the device of the invention the the ultrasonic vibration generator is incorporated in a handpiece to which the solid needle is attached, such as shown in FIG. 1. The needle may furthermore be designed as a single-use disposable needle.

The device according to the invention can be used for open-surgical applications, such as for example during CABG operations. However, the device can also be adapted for endoscopic procedures. To this end, the handpiece preferably incorporates an elongated small-diameter section between the transducer and the solid needle, enabling the needle to reach deep into the body through a small-diameter incision. The device may also be used in robotic surgery systems.

In a further preferred embodiment of the device, the needle is enveloped by a sheath of plastic material through which a lubricant and/or cooling medium may be flushed.

The invention further relates to the use of a device as described for performing direct transmyocardial revascularisation, and to the use of said device for the prevention and/or treatment of angina pectoris.

The invention is further illustrated in the Examples and figures.

FIG. 1 shows a preferred embodiment of the device of the invention. The device comprises a handpiece to which a solid tapered needle is attached (upper panel). The handpiece incorporates an elongated small-diameter section in order to be used for endoscopic procedures. The lower panel shows a preferred embodiment of the attachable solid needle according to the invention, designed for tissue perforation, which is tapered such that during use the ultrasonic vibration waves of the ultrasonic vibration generator are amplified such that cavitation effects occur at the distal tip of the needle.

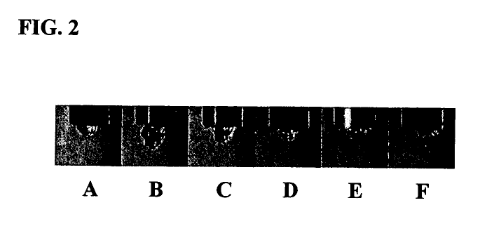

FIG. 2 shows the expanding and imploding cavitation bubble sequence at the distal tip of the needle according to the invention in a liquid environment.

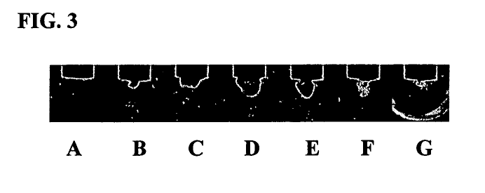

FIG. 3 shows high contrast images of cavitation bubble sequence at the distal tip of the needle according to the invention in a liquid environment with a shock wave visible in the last frame (G).

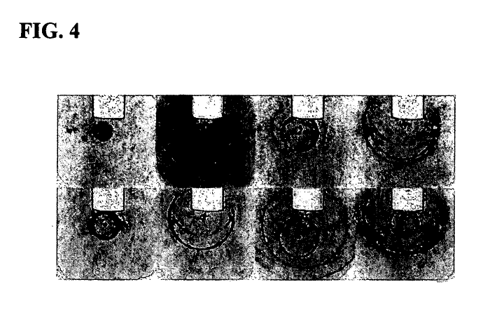

FIG. 4 is a close up view of multiple shock waves underneath the needle tip (inverse Schlieren image).

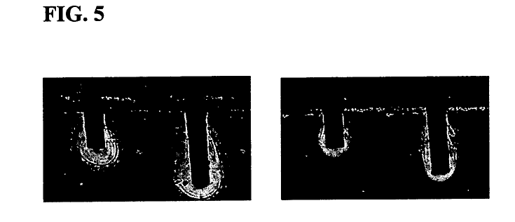

FIG. 5 is a visualisation of the thermal effects of ultrasonic needle perforating in transparent gel at 0.3 mm/s (left) and at 1.8 mm/s (right).

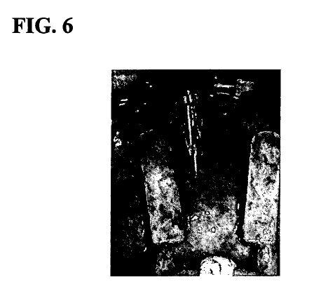

FIG. 6 shows an image of the device according to the invention perforating the myocardium which is locally immobilised with the “octopus” system.

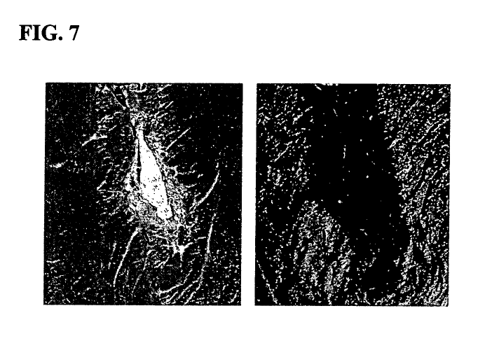

FIG. 7 is a H&E stained histological sample of a channel created with the device according to the invention in porcine myocardium, using regular transmission microscopy (left), and polarised light (right). At the bottom, a clot of cell debris can be appreciated.

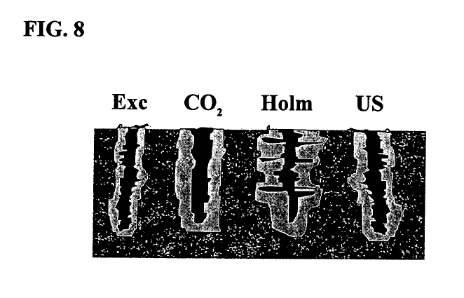

FIG. 8 shows the channel characteristics (fissures and thermal damage zone) of channels perforated by the Excimer, CO₂ and Holmium laser and the device according to the invention.

### EXAMPLES

### Example 1

The device characteristics of the device according to the invention were investigated in vitro and in vivo and compared to laser systems currently used for TMR.

Mechanism of Action

The mechanism of action of the device according to the present invention is mainly ascribed to the formation of macro cavitation bubbles. These cavitation bubbles are formed in a fluid-like environment (water, blood, organic tissue). To characterise and understand the working mechanism of the device high speed visualisation techniques were employed (Verdaasdonk et al., SPIE proceedings 3249: 72-84, 1998; Verdaasdonk et al., SPIE proceedings 2391: 165-175, 1995).

The needle was placed in a water bath, and close-up high-speed photographs were taken at 5 μs intervals during the 40 μs motion cycle of the tip (FIG. 2 and 3). Using Schlieren techniques very high contrast images are obtained enabling the visualisation of shock waves (FIG. 3 and 4).

The needle motion in the liquid can be considered best as a cosine function. In the first half period the needle protrudes, whereas the second half period the needle retracts. The frames in FIG. 2 and 3 show the sequence of a cavitation bubble formation and collapse during the retraction period of the needle.

During tip retraction, the cylindrical distal tip moves at a maximum speed of about 20 m/s through a liquid environment. The fluid has difficulty filling the gap that is left behind (frames A to D). This hole is near vacuum. Due to the extreme under-pressure, the surrounding fluid is sucked inward from all directions at the same time (frames D to F). The acceleration of the fluid is tremendous. During this process, fragments or layers of soft tissue near the cavitation bubble are separated from the underlying tissue. When the hole is filled, there will be collapse of fluid near the center of the original gap.

Since this process is usually not symmetrical, so-called jet-streams are formed focusing the momentum of the accelerated fluid at particular positions preferentially at the surface of tissue. The mechanism described can be selective for tissue structure. Soft tissue is easily fragmented. Hard tissue does not give way and therefore amplifies the jet-streams focused on the tissue surface which fragment it locally. Elastic tissue can partly follow the ‘low’ speed part of the expansion and implosions, and deform without breaking, and so stays intact. The extremely high forces during the collapse can also induce shock waves as described below.

The multiple shock waves in FIG. 4 reveal that the cavitation bubble implosion is not symmetrical, resulting in multiple foci of implosion. The original cavitation bubble breaks up in fragments during the implosion process. The implosions take place about 200 μm underneath the needle surface. As the shock waves grow, one can see that they rebound from the surface of the titanium needle tip. Since the shock waves move at speeds over 1500 m/s, they dissipate in only a few microseconds. The presence of cavitation bubbles and shock waves has also been confirmed in a transparent polyacrylamide gel with and without a biological tissue boundary.

In addition to the cavitation effects, ‘ordinary’ mechanical effects are also present. When the titanium tip is in direct contact with the tissue, it will push against the tissue with extreme forces. The relatively sharp rim (90 degrees) of the tip can cut easily into the soft tissue. Biological hard tissue, which does not deform at all due to the bubble implosions in this case, is no competition for a metal like titanium. The metal tip will pound on the tissue surface shattering it to small pieces.

During vibration, the tip will move at high speed through the fluid and tissue. Tissue in contact with the front and side surface of the needle will not follow the motion. Due to friction and mechanical resistance, energy is dissipated, heating the tissue in the regions of contact. The temperature rise can be considerable, due to the high speed motion and the friction. In the design of the tip, friction may be accounted for using the irrigation fluid from the plastic protective sheath passing over the needle, which further may act as a lubricant and cooling medium.

The presence and extent of thermal effects was visualised using a special optical method based on color Schlieren imaging techniques. Temperature increases in an transparent model tissue are visualised as colours of the rainbow, where red represents the highest temperature. FIG. 5 shows the temperature effects around the tip during penetration into tissue. The temperature rise is highest at the tip due to energy dissipation of the cavitation bubbles and friction. During tissue penetration a ‘thermal tail’ is left behind while the tissue is cooling down. The extent of thermal effects depend on the needle penetration speed. The left panel in FIG. 5 shows the thermal effects while penetrating at 0.3 mm/s and the right panel at 1.8 mm/s. The thermal effects are also dependent on the power applied. By activating the tip sequentially, as one would do during ECG triggering, the thermal effects would decrease due to sufficient cooling between the activations.

### Example 2

In vivo Application of the Device According to the Invention

The device according the invention was tested in a pig model in comparison to laser modalities. The handheld ultrasound device was tested for intra-operative surgical application during a coronary artery bypass graft (‘CABG’) procedure.

The animals underwent surgery according standard protocols approved for animal experiments. After thoracotomy, the heart was locally immobilised using the ‘octopus’ stabilisation techniques for off-pump bypass surgery (developed in Utrecht and distributed by Medtronic). Between the ‘tentacles’ of the ‘octopus’ holes were drilled while recording close-up images with a video system (FIG. 6). During tissue penetration, the ECG was recorded to identify any potential arrhythmias. The animal was sacrificed after 1 hour, and the myocardium containing the channels was taken out and fixated for histological examination. FIG. 7 shows the myocardium with H&E staining in normal light (left) and polarised light (right). Along the channel wall, small fissures can be appreciated, and the zone of thermal injury can be seen up to 500 μm from the channel in polarised light. These channels are comparable to the channels created with the 308 nm excimer laser. A mass of totally pulverised tissue can be appreciated at the bottom of the channel. Individual cells can no longer be discriminated, although cell nuclei are present in the pulp material. This clearly shows how effectively the tissue is ‘ground’ to pieces by the forces of the cavitation bubbles. Also the zone of thermal damage can be well distinguished due to the loss of polarisation of the muscle fibers.

FIG. 8 shows the characteristics of channels created by lasers obtained in another study comparing the different laser modalities and the device according to the invention. The shape and size of the channels resemble the channels obtained with the excimer laser most. The in vivo experiments, however, showed a thermally damaged zone extending approximately 500 μm from the channel wall. This is more comparable to the thermal damage induced by the Holmium laser. As illustrated in FIG. 5, the thermal effects are related with various parameters such as penetration speed. It is not known to what extent the thermal and mechanical damage contributes to the formation of capillaries in the myocardium.

The present invention thus provides a device which induces similar tissue effects as the TMR laser systems, utilizing relatively simple and conventional surgical ultrasound equipment connected to the novel solid titanium needle. By providing a simple and much more cost-effective approach to TMR, transmyocardial revascularlisation may be adopted more rapidly, and may become available in centers throughout the world to improve the treatment of patients with coronary artery disease.

1. Device for perforating tissue, comprising an ultrasonic vibration generator coupled to an attachable solid needle.

2. Device as claimed in claim 1, wherein the needle is shaped in a conical taper.

3. Device as claimed in claim 1 or 2 wherein the solid needle is machined from titanium.

4. Device as claimed in claim 1, 2 or 3 wherein the distal tip diameter of the needle is approximately 1 mm.

5. Device claimed in any one of claims 1-4, wherein, during use, the ultrasonic vibration generator generates a vibration with a frequency in the range of 20-60 kHz.

6. Device as claimed in claim 5 wherein the ultrasonic vibration frequency is 23 kHz.

7. Device as claimed in claim 5 wherein the ultrasonic vibration frequency is 35 kHz.

8. Device as claimed in any one of claims 1-7 wherein the ultrasonic vibration generator is incorporated in a handpiece to which the solid needle is attached.

9. Device as claimed in any one of claims 1-8 wherein the ultrasonic vibration generator comprises a piezo-electric or ferromagnetic transducer.

10. Device as claimed in claim 8 or 9 wherein the handpiece incorporates an elongated small-diameter section between the transducer and the solid needle.

11. Device as claimed in any one of claims 1-10 wherein the needle is enveloped by a sheath of plastic material.

12. Use of a device as claimed in any of claims 1-11 for performing transmyocardial revascularisation.

13. Use of a device as claimed in any of claims 1-11 for the prevention and/or treatment of angina pectoris.

14. Method for revascularising myocardial tissue by creating channels in the myocardial tissue using a device as claimed in claims 1-11.
